(19) United States
(12) Patent Application Publication
(10) Pub. No.: US 2024/0420290 A1
(43) Pub. Date: Dec. 19, 2024

(54) Face Alignment and Normalization For Enhanced Vision-Based Vitals Monitoring

(71) Applicant: Samsung Electronics Co., Ltd., Suwon-si (KR)

(72) Inventors: Korosh Vatanparvar, San Jose, CA (US); Jeremy Speth, South Bend, IN (US); Jicheng Li, Mountain View, CA (US); Li Zhu, Saratoga, CA (US); Nafiul Rashid, San Jose, CA (US); Migyeong Gwak, Santa Clara, CA (US); Jilong Kuang, San Jose, CA (US); Jun Gao, Menlo Park, CA (US)

(21) Appl. No.: 18/677,228

(22) Filed: May 29, 2024

Related U.S. Application Data
(60) Provisional application No. 63/472,787, filed on Jun. 13, 2023.

(51) Int. Cl. G06T 5/70; A61B 5/00; A61B 5/0205; A61B 5/024; A61B 5/11; G06T 5/20; G06T 5/60; G06T 7/00; G06T 7/246; G06T 7/73; G06V 10/776; G06V 40/16

(52) U.S. Cl. CPC G06T 5/70; A61B 5/0205; A61B 5/02416; A61B 5/1128; A61B 5/7207; A61B 5/7267; G06T 5/20; G06T 5/60; G06T 7/0012; G06T 7/248; G06T 7/74; G06T 2207/10016; G06T 2207/20004; G06T 2207/20081; G06T 2207/20084; G06T 2207/30201; G06V 10/776; G06V 40/165

(57) ABSTRACT

In one embodiment, a method includes accessing a video of a user's face. The method further includes accessing, for each image frame in the video, (1) one or more facial landmarks determined by a facial landmark detection (FLD) model and (2) a corresponding determined position in the image for each facial landmark. The method further includes determining, based on the one or more facial landmarks and corresponding positions, a motion of the user's face in the captured video; extracting, from the determined motion of the user's face, a corrected motion signal of the user's face; adjusting, based on the extracted corrected motion signal of the user's face, the positions of one or more facial landmarks in the image frames; and determining, based at least in part on the adjusted positions of the facial landmarks in the sequential images of the video, one or more vital signs of the user.

### PRIORITY CLAIM

This application claims the benefit under 35 U.S.C. § 119 of U.S. Provisional Patent Application No. 63/472,787 filed Jun. 13, 2023, which is incorporated by reference herein.

### TECHNICAL FIELD

This application generally relates to face alignment and normalization for enhanced vison-based vitals monitoring.

### BACKGROUND

Vital signs such as heart rate (HR), respiration rate (RR), oxygen saturation (SpO2), heart rate variability (HRV), blood pressure (BP), and stress index (SI), have long been considered to be important indicators of a person's health. Monitoring these vital signs has traditionally been performed by sensors that contact a person. For example, a pulse oximeter clips to a person's finger and measures the reflection or absorption of light from the person's tissue to estimate vitals including heart rate and blood oxygen levels. Measuring the amount of light absorbed or reflected by human tissues is known as photoplethysmography (PPG).

Contactless or remote sensors can also be used to measure vital signs. For example, remote PPG (rPPG) typically involves capturing images of a person's skin and determining, from these images, changes in light absorbed by or reflected from human tissue. These changes can then be related to vital signs. For example, changes in blood volume in a blood vessel caused by pressure changes due to heartbeats can influence how a given frequency of light is absorbed by the blood vessel, and these changes can be used to determine related vital signs.

### BRIEF DESCRIPTION OF THE DRAWINGS

FIG. 1 illustrates an example method that improves face detection and tracking for vital-sign monitoring.

FIG. 2 illustrates an example implementation of the example method of FIG. 1, among other techniques described herein.

FIG. 3 illustrates an example of an extracted, corrected motion signal for a facial landmark corresponding to the outer corner of the right eye, using adaptive filtering.

FIG. 4 illustrates an example computing system.

### DESCRIPTION OF EXAMPLE EMBODIMENTS

Vision-based health-monitoring systems are often more convenient and less intrusive than corresponding (but often more accurate) invasive methods. For instance, remote PPG (rPPG) techniques are more convenient and less intrusive than contact-based PPG methods. For example, rPPG techniques use ubiquitous devices, such as a camera, that are commonly found in everyday environments, while contact-based methods use less common, specialized devices, such as a pulse oximeter. In addition, rPPG measurements involve capturing images of a subject, which is less intrusive and less uncomfortable than wearing a device, such as a pulse oximeter that severely limits use of the hand, or wearing a chest band. As a result, rPPG measurements can effectively be made much more frequently than PPG measurements, enabling more frequent monitoring of a person's vital signs. For example, rather than having pulse rate or blood oxygen monitored only each time a person visits a medical facility and wears a pulse oximeter, rPPG enables monitoring of pulse rate or blood oxygen (among other vital signs) as the person goes about their tasks in an environment that includes a camera for capturing images of the user, typically the user's face.

However, rPPG signals suffer from various artifacts that tend to decrease the accuracy of a resulting vital sign determination relative to a contact-based approach. One major source of inaccuracy is relative motion of the subject: the pulse signal is typically very subtle compared to other dynamics in video, and if the subject moves, the rPPG signal can be challenging or impossible to recover from the video data. Additionally, noisy camera sensors can cause pixel changes over time which are not representative of observed environmental changes.

rPPG typically relies on reflections off of skin on a person's face to make rPPG measurements, and therefore face detection and tracking is a key component of the rPPG pipeline. Performance limitations and inaccuracies in face detection can therefore corrupt the subtle timeseries rPPG signal. In addition, while certain face-tracking approaches quantify the error in terms of the face detection performance for static images, in rPPG applications accurate tracking of a person's face over a sequence of images is critical to ensuring a robust and accurate rPPG signal. While the discussion above relates to vision-based vital monitoring systems that specifically use rPPG signals, this disclosure contemplates that accurate face detection and tracking can be used for other vision-based vital monitoring systems.

This disclosure describes techniques that improve the reliability and accuracy vision-based vital monitoring systems (e.g., rPPG) by improving the accuracy of face detection and tracking in video of the user's face for estimating one or more vitals of the user.

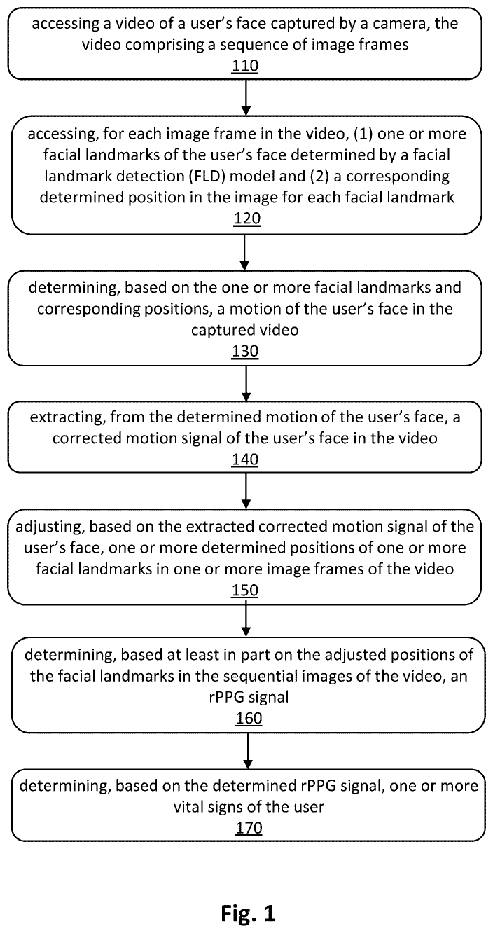

FIG. 1 illustrates an example method that improves face detection and tracking for vital-sign monitoring. Step 110 of the example method of FIG. 1 includes accessing a video of a user's face captured by a camera, where the video is a sequence of image frames. The camera may be any suitable camera, such as a camera of a smartphone, laptop, TV, webcamera, security camera, etc. For example, a user may be sitting in front of their computer, and a camera facing the user can capture video of the user's face. However, the user's face may not be oriented in the same plane as the camera sensor, and the user's face may move throughout the video segment, i.e., the actual position of the user's face may change between and across image frames in the video. Step 110 may include accessing the video directly from the camera (e.g., step 110 may be performed by the client device that contains the camera), or step 110 may be performed by accessing the video from a memory of another device (e.g., the camera may send the video to, or store the video on, a particular device such as a server device or a client device, and step 110 may include receiving the video from such device or downloading the video from that device).

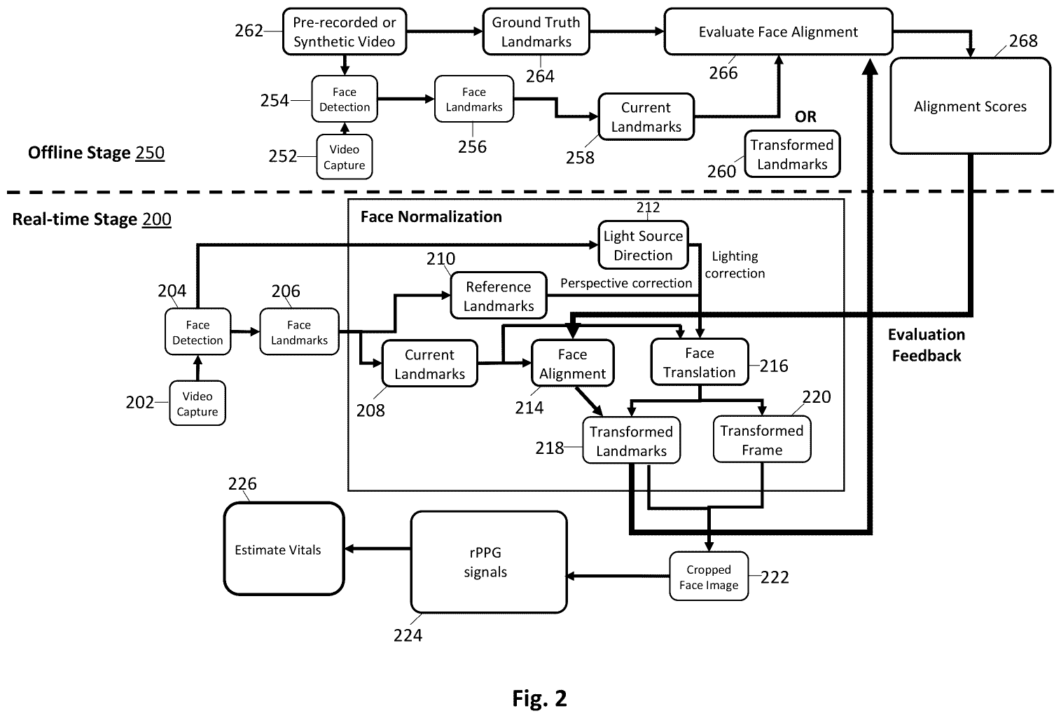

Step 120 of the example method of FIG. 1 includes accessing, for each image in the video, (1) one or more facial landmarks of the user's face, as determined by a facial landmark detection (FLD) model and (2) a corresponding determined position in the image for each facial landmark. A facial landmark detection model detects the presence of a face in an image by detecting facial landmarks (e.g., nose, forehead, etc.) along with the positions of the face and facial landmarks in the image. For example, an FLD model may determine the pixels in an image frame that correspond to e.g., the user's nose, or to the outer corner of an eye, and thereby may determine the pixel location of such facial landmarks in that image frame. Many different FLD models exist, and this disclosure contemplates that any suitable FLD model may be used for detecting the presence and location of a user's face and constituent facial landmarks in an image. FIG. 2 illustrates an example implementation of the example method of FIG. 1, among other techniques described herein. In FIG. 2, face landmarks 206 are determined by an FLD model during the real-time vital-sign estimate process 200 for determining a user's current vital signs.

Step 130 of the method of FIG. 1 includes determining a motion of the user's face in the captured video. A perfect FLD model would accurately and precisely detect and track a user's face and corresponding facial landmarks across a sequence of images. However, in practice the position of a user's face and facial landmarks as detected by an FLD model may deviate from the actual ground truth of those positions in an image. Moreover, the positional movement of a user's face and corresponding facial landmarks as detected by an FLD model across a sequence of images may not correspond to the actual movement of the user's face across those images, and as a result, this noise in the position signal propagates throughout a video and decreases the accuracy of the resulting rPPG signal and vital signs determined from the video. In other words, the position of a user's face over time as determined by an FLD model attempting to track the user's facial landmarks throughout the video may deviate from the actual position of the user's face over time in the video. In practice, noise in the positional signal can arise from multiple factors, including the FLD model, video encoding, camera sensor noise, light variation, and so on.

The positional variation in a user's face across images as determined by an FLD model occurs as the FLD model perceives changes in the position (e.g., pixel locations) of facial landmarks in the image. When comparing the position of facial landmarks in each frame as determined by an FLD model to the actual, ground-truth position of those landmarks, one typically observes a very high-frequency noise in the FLD-determined motion signal that does not correspond to the user's actual movement. Typically, a user's movement is a relatively slow (low frequency) event. In addition, there may be temporal “spikes” in the FLD-determined motion signal for one or more facial landmarks due to noise in the FLD model and in the video recording, among other sources. Since an rPPG signal is generated by tracking pixel values in the image, inaccurate movement of the pixels used by RGB signal can improperly change the baseline, resulting in significant noise in the rPPG signal.

Step 140 of the example method of FIG. 1 includes extracting, from the determined motion of the user's face, a corrected motion signal of the user's face in the video. The techniques described herein approximate the actual motion of a user's face from the noisy motion signal determined by the FLD (and accessed in step 130). In practice, the motion signal extracted from FLD should have a relatively low frequency spectrum, which naturally corresponds to the behavior of users sitting in front of camera. In these cases, a user's movement mainly consists of sudden movements of head (i.e., short, relatively drastic movements) and a relatively slow movement of head induced by breathing (i.e., a relatively slow, low-amplitude movement).

This disclosure contemplates that multiple different approaches may be used to extract a corrected motion signal of a user's face, thereby approximating the actual motion of the user's face. For example, processing the motion signal by filtering a moving average of the motion signal (of the entire face or of the position of one or more landmarks) using one or more low pass filters reduces the high-frequency noise in the FLD-determined motion signal. In this approach, historical samples are considered and averaged to produce the current motion signal. An alpha parameter a defines how much to smooth the signal, adjusting the weight given to current sample vs historical samples. For instance:

$${\hat{X}}_{1} = {X}_{1} \\ {\hat{X}}_{i} = α {X}_{i} + ( 1 - α ) {\hat{X}}_{i - 1} , i \geq 2$$

where \hat{X}ᵢ is value of filtered motion signal at index i, calculated based on original motion signal Xᵢ and previous filtered samples \hat{X}ᵢ₋₁. This filter approach can work well when a user is stationary and does not make sudden movements. A user's head moves relatively slowly most of the time, and therefore low-pass filters can be effective in such instances. However, the extracted corrected motion signal should also capture sudden shifts in the user's face due to actual, sudden movement of the user's body or posture; otherwise, filtering out such sudden movements can introduce significant errors in vital-sign estimates due to the mismatch between a user's actual and determined facial position.

Unlike a static low-pass filter, one or more dynamic filters such as one-euro or Kalman filters adaptively change their filtering behavior by taking into account the speed of the motion signal, and such adaptive filters may be used to process the FLD-determined motion signal for the user's face and/or one or more facial landmarks. In adaptive filters the alpha parameter a is not constant. Instead, it is adaptive and dynamically computed using information about the rate of change (speed) of the signal. The adaptive smoothing factor aims to balance the jitter vs. lag trade-off; this approach may be particular well suited for extracting a corrected motion signal of a user's face from the FLD-determined motion of the user's face, since face-tracking applications are sensitive to jitter at low speeds and more sensitive to lag at high speeds. The smoothing factor may be defined as:

$$α = \frac{1}{1 + \frac{τ}{{T}_{e}}} \qquad ( 1 )$$

where τ is time constant computed using the filter cutoff frequency

$$( τ = \frac{1}{2 π {f}_{c}} )$$

and Tₑ is the sampling period computed from the time difference between the samples. Thus, step 140 may include filtering the FLD-determined motion signal using a dynamic filter with an adjustable alpha parameter a, removing spurious high-frequency signals from the data while still tracking motion due to sudden movements of the user's face.

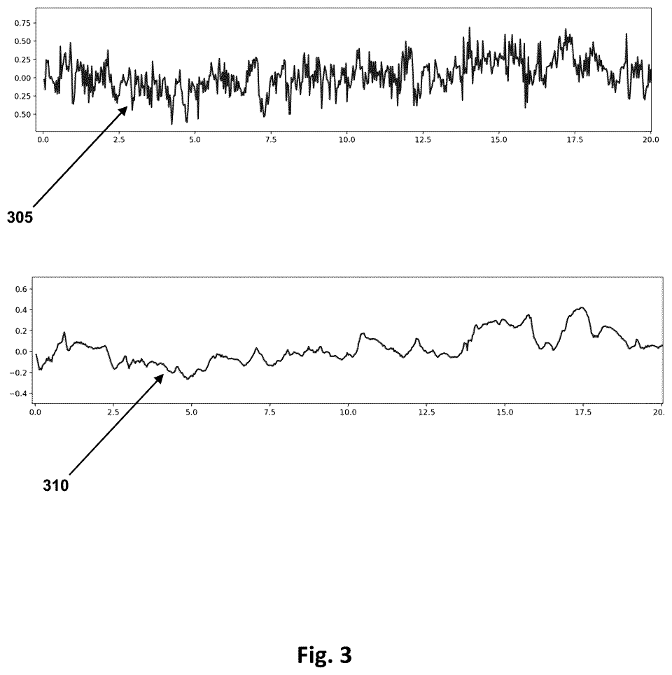

FIG. 3 illustrates an example of an extracted, corrected motion signal 310 for a facial landmark corresponding to the outer corner of the right eye using adaptive filtering. The motion in the video of the outer corner of the right eye as determined by the FLD is illustrated by motion signal 305. As illustrated in FIG. 3, the motion of the outer corner of the right eye as determined by the FLD model has much greater jitter than corrected motion signal 310, as the dynamic filter removed motion due to noise in the FLD (and that would correspond to unrealistic or and unlikely actual motion) while retaining (1) physiological motion (e.g., periodic motion of the face due to breathing) and (2) more pronounced and non-periodic (e.g., voluntary) movements of the user's face.

Instead of, or in addition to, a filter-based approach, machine-learning and deep-learning methods may be used to extract a corrected motion signal from the FLD-determined signal using a trained face-alignment model. For example, a user's head motion may be tracked by (1) using FLD to determine the positions of facial landmarks, and (2) using one or more ground-truth motion/orientation sensors (e.g., in earbuds or head wearables) concurrently with the FLD determinations. A face alignment model may be trained by pairs containing (1) FLD determinations (which may be treated as the noisy data) and (2) concurrent motion data as determined using the ground-truth sensors (which may be treated as the target data). A comprehensive dataset of such concurrent pairs across various levels of user motion may be used to train a face-alignment model, and this trained model may then be used at runtime to output a corrected motion signal for the user's face. Examples of particular face-alignment models include a CNN-based models or other alternatives such as RNN that can capture the temporal and frequency information of the motion signal. In particular embodiments, a ML or DL face alignment model may be able to correct motion in a wider variety of motion types than a filter-based approach.

FIG. 2 illustrates an example implementation of the example method of FIG. 1, among other techniques described herein. As illustrated in FIG. 2, a camera captures a sequence of images of a user's face in video capture step 202. A face-detection model performs face detection 204 to determine, for each image frame, the location of the user's face in the image. An FLD model (which may be part of the overall face detection model) determines the presence and position of face landmarks 206 in each image. These landmarks are the current landmarks 208 as determined by the FLD model. In the conventional approach, these landmarks would be used to determine how and where to crop the image to the just the user's face—e.g., to obtain a cropped face image 222—and therefore, inaccuracies in the position of current landmarks 208 results in inaccurate copped face images 222 and therefore inaccurate rPPG signals 224 and vital-sign estimates 226. However, the techniques described herein perform a face alignment 214 to obtain transformed landmarks 218, which are the current landmarks 208 with their position corrected, if necessary, by face alignment model 214. For example, as described above with respect to steps 130 and 140, the face alignment model 214 may filter a facial-motion signal of the user and/or may alter the position of the current landmarks 208 by a trained ML or DL face-alignment model to correct the position of the landmarks, thereby obtaining transformed landmarks 218. This is also reflected in step 150 of the example method of FIG. 1, which includes adjusting, based on the extracted corrected motion signal of the user's face, one or more determined positions of one or more facial landmarks in one or more images of the video.

Step 160 of the example method of FIG. 1 includes determining an rPPG signal, based at least in part on the position of facial landmarks in sequential images of the video. For instance, in the example implementation of FIG. 2, cropping 222 occurs based on the determined location of the user's face, which in turn depends on the determined location of facial landmarks. Cropped face images 222 are based on the position-corrected transformed landmarks 218, and therefore the image-cropping in each image frame of the video accurately represents the ground-truth position of the user's face. rPPG signals 224 are determined based on the color values of the pixels in the portion of the image determined to correspond to the user's face (or to a region of the user's face), i.e., the pixels in cropped face image 222.

Step 170 of the example method of FIG. 1 includes determining, based on the determined rPPG signal, one or more vital signs of the user. As explained above, such vital signs may include SpO2, heart rate, respiratory rate, or blood pressure, among others.

Users can freely move their head in front of a camera while video is being captured of the user, and as a result, the user's face and facial landmarks may appear in different x,y pixel locations across images in a video. As discussed above, varying locations of landmarks may create difficulties in accurately tracking landmarks across images, and therefore may reduce the accuracy of vital-sign estimations based on the video. Moreover, when a user changes the orientation of their face (e.g., rotates their face), this alters the relationship between facial landmarks (e.g., the x,y distance in the image between the left check and the right check, the relative sizes of landmarks, etc.).

Particular embodiments apply a transformation to image frames so that approximately similar x, y values in the image correspond to the same facial landmarks across images. For instance, face translation module, such as face translation model 216 of the example implementation of FIG. 2, utilizes the landmarks identified on the face along with a reference image to translate the face images. In one approach, the first frame in a sequence of images is utilized as the reference frame. In another approach, a predefined reference frame of ideal reference landmarks can be defined for image translation.

Facial movements such as scaling, rotation, and perspective changes can impact how a face appears in images. Methods such as homography can help correct these variations, especially to normalize the perspective of the image. For instance, in the case of various perspectives, as the angle of the face changes, certain regions of the face become less visible, and other regions scale up and become more visible. One or more homography matrices can be used to provide one or more transformation matrices between two planes (i.e., between two perspectives), thereby adjusted a mesh grid defining the face and the facial landmarks. In other words, a face may not be directly inline with (looking straight at) a camera, and frame translations can translate the perspective of the entire image as the image would appear if the user were looking straight at the camera.

Particular embodiments use homography to translate FLD landmarks identified in each face image to the reference landmarks identified in a reference image frame. Particular embodiments use the first frame in a capture video as the reference. However, because the first frame may not be an ideal reference, other embodiments use a different reference frame, as described below.

For example, a “reference face” can be defined such that the skin pixels in all regions are equally exposed with more focus on regions where more blood perfusion exists. In particular embodiments, a reference brightness level can be defined for various regions of the face such that not only is the face perspective translated, but the light intensity on the face across images of the face is also translated to the light intensity of the reference frame. In particular embodiments, the direction of light can be estimated based on the reflection on the skin. The approximate angle and direction of light with respect to the face can be used to normalize the light brightness on the skin, correcting various levels of shading on the skin, and ensuring that the brightness distribution across regions of the user's face is consistent throughout a video. For instance, if person's forehead is brighter than either check in a reference image, and each check has an equivalent brightness, then this intensity profile (along with the specific intensities of those regions) may be applied to other frames in the video.

Light correction across images may be done by translating the face image to the reference “light-normalized” frame. This can be done similar to homography by using a transformation matrix that maps 2D light intensity at x, y locations of the face to the reference face image. The XY plane can be quantized into smaller regions for better translation of light intensity. In another approach, a CNN model is trained and then used to map both landmarks (for perspective correction) and light intensity at the same time, taking as input a 2-D image frame and generating as output the translated frame.

In particular embodiments, instead of utilizing landmarks from the first frame of a video as the reference landmarks, reference landmarks may be ground-truth landmark locations determined by other sensors (e.g. an accelerometer in earbuds, in a head-worn device, etc.) that a user may wear while being recorded by a camera. The ground truth landmark locations can be used as the reference landmarks to identify the facial alignment or mis-alignment. As another example, a reference frame may be a frame in which the user is looking straight at the camera (or nearly straight at the camera), and subsequent frames may be adjusted based on that reference frame.

FIG. 2 illustrates an example implementation of transforming one or more image frames by applying a face translation. In the example of FIG. 2, reference landmarks 210 are provided to face translation module 216. As discussed above, reference landmarks may come from, for example, a first video frame, or from ground-truth reference data provided by a coincidently worn sensor, etc. The face translation model 216 translates each non-reference frame based on the perspective and/or orientation of the reference frame, and these translations adjust the position of current landmarks 208 to arrive at transformed landmarks 218. In particular embodiment, transformed landmarks 218 include both the noise-reducing positional adjustments made by face alignment 214 and the orientation/perspective frame transformations made by face translation model 216.

As illustrated in FIG. 2 and as described above, a reference light-source direction 212 may be determined and provided to face translation model 216, so that the lighting in non-reference frames is transformed to the lighting in the reference frame. Step 212 may be determining a face brightness map in the reference frame. Applying the face translation model to a frame results in the transformed image frame 220, which corrects the light intensity across the face, thereby improving accurate extraction of a physiological signal throughout the video in the presence of face movement and light variations caused by the movement.

As illustrated in FIG. 2, particular embodiments may perform both face alignment and face translation to improve video frames for subsequent vital-sign estimation. For instance, transformed landmarks 218 may include, for any given any image in the video, both positional adjustments due to the corrected motion signal from face alignment model 214 and also positional adjustments due to a perspective correction as determined by face translation model 220. For instance, face alignment model 214 adjusts the position of landmarks by removing noise from the FLD-determined motion signal, while face translation model 216 adjusts the positions of landmarks through perspective-adjustment of an image based on a reference frame. Face translation model 216 may also output a transformed frame by adjusting the lighting intensity in regions of the frame, based on the intensities in a reference frame. Transformed frame 220 may include the lighting-intensity adjustments for that frame. In particular embodiments, lighting adjustments may occur first, and then positional adjustments may be made on the lighting-adjusted frame. In other embodiments, positional adjustments may occur first, or the two operations may be performed in parallel.

As explained above, differences between the facial location determined by an FLD model and the actual face location can introduce inaccuracies in the subsequent steps of determining rPPG signals and estimating vital signs, and removing these inaccuracies is an important challenge in remote vital sign detection. An approach for improving the accuracy of FLD determinations involves quantifying the error in face alignment output by an FLD, which can be used to evaluate and improve the FLD and to select among available FLD models for determining facial landmarks, so that the most accurate model is selected to perform landmark detection.

This disclosure describes techniques for determining quantitative scores of landmarks quality generated by a particular face model. These quantitative scores are correlated with performance of vital monitoring, and as explained below, these scores can be calculated on the output of an FLD model and/or on the output of such models after correction, such as after correction by one or both of a face alignment model and a face translation model, both of which are described above. These quantitative scores facilitate determinations of whether to further improve landmark detection, and inform the trade-off between complexity, accuracy, and computation time.

FLD models include both deep learning-based and traditional computer vision-based models. Deep learning-based models, such as MobileNet, DeepFace, or BlazeFace use convolutional neural networks (CNNs) to learn features from raw image data. In contrast, traditional computer vision-based models, such as Haar Cascades or Eigenfaces, use hand-crafted features, such as edges and corners, to detect faces. While deep learning-based models have shown promising results relative to computer-vision based models, they often require large amounts of training data and computational resources.

Noise in landmarks generated by FLD models can arise from various sources, including variations in background objects, noise in input sensors, face occlusion, or environmental conditions. These factors can cause fluctuations in the position and orientation of facial landmarks, leading to inaccuracies in the detection and tracking of faces. To mitigate the effects of noise, various techniques can be employed, such as data augmentation, regularization, and post-processing. These techniques can help to improve the robustness and accuracy of FLD models, making them more reliable in real-world applications.

Current metrics for evaluating face alignment typically adopt a “static” approach that considers all facial landmarks collectively within a single frame. Such techniques include Mean Euclidean Distance (MED), Normalized Mean Error (NME), Failure Rate (FR), and Area Under the Curve (AUC). These metrics calculate the average distance between predicted and ground truth facial landmarks, normalize this distance with a reference length to account for face size variations, evaluate the detector's failure rate in locating facial landmarks within a threshold, and consider the trade-off between true positive and false positive rates. But in contrast to the static approach, and as explained above, contactless vital-sign estimation requires accurately tracking the trajectory of each landmark in a sequence of images over time, as such estimation requires image data over a period of time (e.g., several seconds). Therefore, the techniques of this disclosure analyze the dynamic changes occurring at each facial landmark for vital sign monitoring. In other words, the techniques described herein evaluate and quantify facial alignment over time, not merely in a single, static image.

FIG. 2 illustrates an example approach for evaluating an FLD model during offline stage 250. As illustrated in FIG. 2, the evaluation in offline stage 250 is separate from real-time stage 200, in which a user's current vital sign(s) are estimated based on captured video.

As illustrated in FIG. 2, pre-recorded or synthetic video 262 is obtained to provide the ground truth facial movements. As explained below, pre-recorded or synthetic video 262 may be static video of a non-moving face or video of a synthetically moving face, e.g., a face moving due to mimicked breathing activity. A dataset of video 262 is collected using a camera placed in front of a reference user at multiple, different distance settings (e.g., 3 ft and 6 ft, which may closely simulate the distance between a camera and a human face in a real-world contactless vital sign collection procedure). As explained below, in particular embodiments the reference user may be a synthetic face, such as a mannequin head.

The dataset of videos includes a set ground truth face locations gt, for example, by adding controllable movement patterns starting with gt₁ (i.e., the initial frame) so that the motion in subsequent frames gtᵢ is derived consecutively. Therefore, in particular embodiments, obtaining gt₁ is a fundamental step to determine the ground-truth face location for a video of a moving face in a video database. In such embodiments, each video has a gt estimation stage and an evaluation stage, where the gt estimation stage occurs at the very beginning of a video to estimate gt₁. Acquiring the ground truth for the subsequent frames differs based on whether the video (e.g., video 262) is a static video of a still user's face or a dynamic video, in which the reference user's face is moving.

For static videos, video collection (e.g., to generate video 262) involves placing a camera in front of a synthetic user's head (e.g., in front of a mannequin head) to ensure absolute facial immobility. While the face is immobile, face-detection jitter still happens due to noise from multiple sources such as cameras, sensors, illumination, and the video encoder. A static video is recorded for a predetermined length, e.g., 30 seconds long, and may include a 3-second ground truth estimation stage and a 27-second evaluation stage. To obtain gt₁ for a static video, particular embodiment average facial detections among all frames for the first 3 seconds of video recording (i.e., the first n frames corresponding to the first 3 second of video recording). Since the face is truly motionless, gt₁ is also the ground truth for all the following frames, i.e., gtᵢ=gt₁, i∈{2, . . . , N}, where N is the number of images in the full video recording (e.g., in the full 30 seconds of recorded video).

For dynamic videos that include ground-truth locations of a moving face, the movement may be synthetically introduced, e.g., to mimic the movement of a user's face due to upper-body movements from breathing. Particular embodiments may create a dynamic video by applying motion patterns extracted from real, human breathing patterns to a template mannequin face. A motion pattern refers to the proxy of horizontal and vertical motion on the x-axis and y-axis denoted as [Δᵢ=(Δxᵢ, Δyᵢ), i∈{1, . . . , N}]. Therefore, once gt₁ is obtained for a dynamic video, the ground truth for all subsequent frames in that video can be determined by:

$${gt}_{i} = \{ \begin{matrix}{gt}_{1} & i = 1 \\ {gt}_{i - 1} + {Δ}_{i} = ( {x}_{i - 1} + Δ {x}_{i} , {y}_{i - 1} + Δ {y}_{i} ) & i > 1\end{matrix} \qquad ( 2 )$$

where (xᵢ, yᵢ) represents the facial landmarks in the ith frame.

In particular embodiments, creation of a dynamic video has two stages: a ground truth estimation stage (i.e., static stage) and an evaluation stage (i.e., a moving stage). The ground truth estimation stage lasts for a predetermined amount of the initial video, e.g., the first 30 seconds, and consists of static frames of the reference face, with gt being the average of all detection results within this static stage, for example to cancel out noise from the video encoder. The evaluation stage lasts for a subsequent predetermined amount of time, e.g., 60 seconds, and involves evaluating a candidate FLD model based on the ground truth derived using equation 2, above, for the motion stage of the pre-recorded or synthetic video. For instance, with reference to FIG. 2, video capture 252 of a reference user (e.g., a mannequin head) may be made a camera. An FLD model may perform face detection 254 to extract face landmarks 256, and the set of landmarks and corresponding positions are defined as current landmarks 258. Meanwhile, a prerecorded or synthetic video 262 is used to create ground truth landmarks 264 as described above, and the FLD-detected landmarks 258 are evaluated against the ground-truth landmarks 264 for each frame to perform an evaluation 266 of the FLD model's face alignment. As illustrated in FIG. 2, in particular embodiments, the evaluation 266 may be performed on translated landmarks 260 (which may be created as described above with reference to transformed landmarks 218 in the real-time stage 200) rather than on the raw output 258 of the FLD model, for example to evaluate the combination of both the FLD model and the subsequent alignment techniques (e.g., alignment model 214 of FIG. 2).

Evaluations 258 are made using one or more alignments scores 268. Three example scoring metrics are described below, each capturing a different aspect of face alignment assessment. For the purposes of explaining example scores, consider five facial landmarks: the four corners of a face bounding box (bb)—top-left (tl), top-right (tr), bottom-left (bl), bottom-right (br), and a nose landmark representing the face center (ct). The location of each landmark is represented by a 2D coordinate (x, y). A video v is composed of N frames, for each frame i, there is a detection result detᵢ=[tlᵢ, trᵢ, blᵢ, brᵢ, ctᵢ] and a ground truth gtᵢ=[gtlᵢ, gtrᵢ, gblᵢ, gbrᵢ, gctᵢ]. The indices for “det” and “gt” start at 1 for annotation purposes. For each of the three example evaluation metrics (or scoring metrics) described below, a smaller value indicates a better performance.

One example scoring metric is circular radius, which is defined as the maximum Euclidean distance between detected and ground truth facial landmarks. Therefore, the circular radius can be represented by:

Circular Radius=max(dist(gtᵢ[j],detᵢ[j]))
where i∈{1, N}, j∈{1, n}, and dist(.) stands for Euclidean distance. The circular radius scoring metric mainly evaluates face misalignment induced by detection outliers.

Another example scoring metric is mean offset, which is defined as the average distance between the ground truth and detected facial landmarks. Therefore, mean offset can be represented as:

$$Mean Offset = \frac{1}{N} {\sum }_{i = 1}^{N} {\sum }_{j = 1}^{n = 5} dist ( {gt}_{i} [ j ] , {det}_{i} [ j ] )$$

Mean offset is different from circular radius in that it primarily evaluates the generalization and steadiness of a face detector and the average misalignment among all landmarks over time.

Another example scoring metric is percentage of impacted pixels (PIP). For frame i, let bb_{det}_{ᵢ} be the bounding box provided by the face detector with an area of S_{det}_{ᵢ}, and bb_{gt}_{ᵢ} be the ground truth bounding box with an area of S_{gt}_{ᵢ}. The percentage of impacted pixels is defined as the ratio of pixels that exist exclusively in either bb_{det}_{ᵢ} or bb_{gt}_{ᵢ} to the union of bb_{det}_{ᵢ} and bb_{gt}_{ᵢ}. Therefore:

$$PIP = \frac{1}{N} {\sum }_{i = 1}^{N} \frac{( {bb}_{{det}_{i}} - {bb}_{{det}_{i}} ⋂ {bb}_{{gt}_{i}} ) + ( {bb}_{{gt}_{i}} - {bb}_{{det}_{i}} ⋂ {bb}_{{gt}_{i}} )}{{bb}_{{det}_{1}} ⋃ {bb}_{{gt}_{i}}} \\ \frac{1}{N} {\sum }_{i = 1}^{N} \frac{{bb}_{{det}_{i}} + {bb}_{{gt}_{i}} - 2 \cdot ( {bb}_{{det}_{i}} ⋂ {bb}_{{gt}_{i}} )}{{bb}_{{det}_{i}} ⋃ {bb}_{{gt}_{i}}}$$

Particular embodiments may select an FLD model based on one or more evaluation scores, such as the example scores described above. In particular embodiments, one or more evaluation scores may be used to determine whether a facial alignment model or a face translation model, or both, are performing with suitable accuracy. For example, if the evaluation score(s) indicate that the models are not performing as desired, then additional model training may be indicated, if the model is a ML or DL model.

The techniques described herein may be used in a wide variety of use cases. For example, the techniques may be used to estimate a user's vital signs during a telehealth visit with a medical professional, and video of the user used for the telehealth visit may also be used to estimate the user's vital signs by tracking the user's face. As another example, the techniques described herein for contactless vital-sign monitoring may be used while a user is using or facing a device, such as a TV, laptop, smartphone, etc., that has a camera facing the user, and therefore passive, contactless estimates of the user's vital signs may be made while the user is using the device or is otherwise engaged in other activities. For example, contactless vital-sign estimates may be made for a user while the user is watching TV, working at a computer, scrolling through content on her smartphone, exercising, etc. Cameras may also be deployed on, e.g., airplanes, cars, in hospitals, etc. for contactless vital-sign estimation of subjects in the field of view of the camera. In addition, the user's vital signs may be monitored more frequently and over longer periods of time, generating trend reports of the user's health over time.

In particular embodiments, the techniques described herein may be used to understand a user's behavior and reactions (e.g., emotions based on vital-sign monitoring) to the user's surroundings, such as to content the user is viewing on a TV or smartphone, while tracking the user's face to make contactless vital-sign determinations. The user's reactions may be used to, e.g., provide content recommendations, create highlights (e.g., of an exciting part of a video game the user is playing), surface health anomalies to the user or to a medical professional, etc. The user's reactions may be used to adjust the intensity of user's workout based on the user's physiological signals as determined by face tracking.

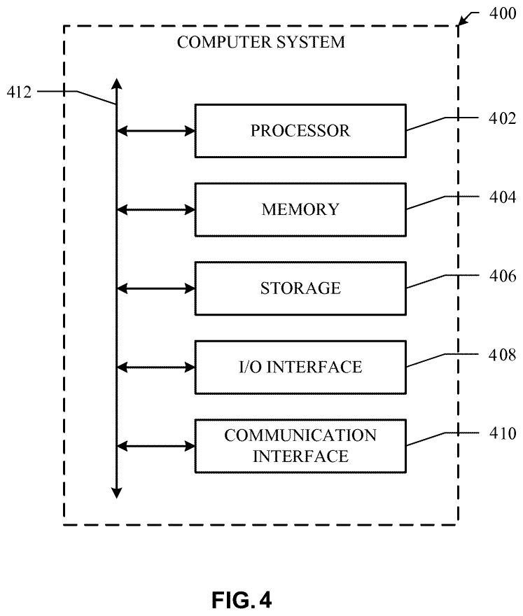

FIG. 4 illustrates an example computer system 400. In particular embodiments, one or more computer systems 400 perform one or more steps of one or more methods described or illustrated herein. In particular embodiments, one or more computer systems 400 provide functionality described or illustrated herein. In particular embodiments, software running on one or more computer systems 400 performs one or more steps of one or more methods described or illustrated herein or provides functionality described or illustrated herein. Particular embodiments include one or more portions of one or more computer systems 400. Herein, reference to a computer system may encompass a computing device, and vice versa, where appropriate. Moreover, reference to a computer system may encompass one or more computer systems, where appropriate.

This disclosure contemplates any suitable number of computer systems 400. This disclosure contemplates computer system 400 taking any suitable physical form. As example and not by way of limitation, computer system 400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 400 may include one or more computer systems 400; be unitary or distributed; span multiple locations; span multiple machines; span multiple data centers; or reside in a cloud, which may include one or more cloud components in one or more networks. Where appropriate, one or more computer systems 400 may perform without substantial spatial or temporal limitation one or more steps of one or more methods described or illustrated herein. As an example and not by way of limitation, one or more computer systems 400 may perform in real time or in batch mode one or more steps of one or more methods described or illustrated herein. One or more computer systems 400 may perform at different times or at different locations one or more steps of one or more methods described or illustrated herein, where appropriate.

In particular embodiments, computer system 400 includes a processor 402, memory 404, storage 406, an input/output (I/O) interface 408, a communication interface 410, and a bus 412. Although this disclosure describes and illustrates a particular computer system having a particular number of particular components in a particular arrangement, this disclosure contemplates any suitable computer system having any suitable number of any suitable components in any suitable arrangement.

In particular embodiments, processor 402 includes hardware for executing instructions, such as those making up a computer program. As an example and not by way of limitation, to execute instructions, processor 402 may retrieve (or fetch) the instructions from an internal register, an internal cache, memory 404, or storage 406; decode and execute them; and then write one or more results to an internal register, an internal cache, memory 404, or storage 406. In particular embodiments, processor 402 may include one or more internal caches for data, instructions, or addresses. This disclosure contemplates processor 402 including any suitable number of any suitable internal caches, where appropriate. As an example and not by way of limitation, processor 402 may include one or more instruction caches, one or more data caches, and one or more translation lookaside buffers (TLBs). Instructions in the instruction caches may be copies of instructions in memory 404 or storage 406, and the instruction caches may speed up retrieval of those instructions by processor 402. Data in the data caches may be copies of data in memory 404 or storage 406 for instructions executing at processor 402 to operate on; the results of previous instructions executed at processor 402 for access by subsequent instructions executing at processor 402 or for writing to memory 404 or storage 406; or other suitable data. The data caches may speed up read or write operations by processor 402. The TLBs may speed up virtual-address translation for processor 402. In particular embodiments, processor 402 may include one or more internal registers for data, instructions, or addresses. This disclosure contemplates processor 402 including any suitable number of any suitable internal registers, where appropriate. Where appropriate, processor 402 may include one or more arithmetic logic units (ALUs); be a multi-core processor; or include one or more processors 402. Although this disclosure describes and illustrates a particular processor, this disclosure contemplates any suitable processor.

In particular embodiments, memory 404 includes main memory for storing instructions for processor 402 to execute or data for processor 402 to operate on. As an example and not by way of limitation, computer system 400 may load instructions from storage 406 or another source (such as, for example, another computer system 400) to memory 404. Processor 402 may then load the instructions from memory 404 to an internal register or internal cache. To execute the instructions, processor 402 may retrieve the instructions from the internal register or internal cache and decode them. During or after execution of the instructions, processor 402 may write one or more results (which may be intermediate or final results) to the internal register or internal cache. Processor 402 may then write one or more of those results to memory 404. In particular embodiments, processor 402 executes only instructions in one or more internal registers or internal caches or in memory 404 (as opposed to storage 406 or elsewhere) and operates only on data in one or more internal registers or internal caches or in memory 404 (as opposed to storage 406 or elsewhere). One or more memory buses (which may each include an address bus and a data bus) may couple processor 402 to memory 404. Bus 412 may include one or more memory buses, as described below. In particular embodiments, one or more memory management units (MMUs) reside between processor 402 and memory 404 and facilitate accesses to memory 404 requested by processor 402. In particular embodiments, memory 404 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 404 may include one or more memories 404, where appropriate. Although this disclosure describes and illustrates particular memory, this disclosure contemplates any suitable memory.

In particular embodiments, storage 406 includes mass storage for data or instructions. As an example and not by way of limitation, storage 406 may include a hard disk drive (HDD), a floppy disk drive, flash memory, an optical disc, a magneto-optical disc, magnetic tape, or a Universal Serial Bus (USB) drive or a combination of two or more of these. Storage 406 may include removable or non-removable (or fixed) media, where appropriate. Storage 406 may be internal or external to computer system 400, where appropriate. In particular embodiments, storage 406 is non-volatile, solid-state memory. In particular embodiments, storage 406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 406 taking any suitable physical form. Storage 406 may include one or more storage control units facilitating communication between processor 402 and storage 406, where appropriate. Where appropriate, storage 406 may include one or more storages 406. Although this disclosure describes and illustrates particular storage, this disclosure contemplates any suitable storage.

In particular embodiments, I/O interface 408 includes hardware, software, or both, providing one or more interfaces for communication between computer system 400 and one or more I/O devices. Computer system 400 may include one or more of these I/O devices, where appropriate. One or more of these I/O devices may enable communication between a person and computer system 400. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 408 for them. Where appropriate, I/O interface 408 may include one or more device or software drivers enabling processor 402 to drive one or more of these I/O devices. I/O interface 408 may include one or more I/O interfaces 408, where appropriate. Although this disclosure describes and illustrates a particular I/O interface, this disclosure contemplates any suitable I/O interface.

In particular embodiments, communication interface 410 includes hardware, software, or both providing one or more interfaces for communication (such as, for example, packet-based communication) between computer system 400 and one or more other computer systems 400 or one or more networks. As an example and not by way of limitation, communication interface 410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 410 for it. As an example and not by way of limitation, computer system 400 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 400 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 400 may include any suitable communication interface 410 for any of these networks, where appropriate. Communication interface 410 may include one or more communication interfaces 410, where appropriate. Although this disclosure describes and illustrates a particular communication interface, this disclosure contemplates any suitable communication interface.

In particular embodiments, bus 412 includes hardware, software, or both coupling components of computer system 400 to each other. As an example and not by way of limitation, bus 412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 412 may include one or more buses 412, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing a video of a user's face captured by a camera, the video comprising a sequence of image frames;
accessing, for each image frame in the video, (1) one or more facial landmarks of the user's face determined by a facial landmark detection (FLD) model and (2) a corresponding determined position in the image for each facial landmark;
determining, based on the one or more facial landmarks and corresponding positions, a motion of the user's face in the captured video;
extracting, from the determined motion of the user's face, a corrected motion signal of the user's face in the video;
adjusting, based on the extracted corrected motion signal of the user's face, one or more determined positions of one or more facial landmarks in one or more image frames of the video;
determining, based at least in part on the adjusted positions of the facial landmarks in the sequential images of the video, an rPPG signal; and
determining, based on the determined rPPG signal, one or more vital signs of the user.

2. The method of claim 1, wherein extracting, from the determined motion of the user's face, a corrected motion signal of the user's face comprises filtering the determined motion of the user's face.

3. The method of claim 2, wherein filtering the determined motion of the user's face comprises applying a dynamic filter with an adaptive alpha parameter a that is based on the rate of the change of the determined motion.

4. The method of claim 3, wherein the alpha parameter
$$α = \frac{1}{1 + \frac{τ}{{T}_{e}}} .$$

5. The method of claim 1, wherein extracting, from the determined motion of the user's face, a corrected motion signal of the user's face comprises:
providing, to a trained face-alignment model, the determined motion of the user's face; and
outputting, by the trained face-alignment model, the corrected motion signal.

6. The method of claim 1, further comprising:
defining a reference image of the user's face, the reference image comprising one or more reference facial landmarks;
determining, for each of one or more subsequent frames in the sequence of image frames, a landmark difference between the facial landmarks in that frame and the corresponding reference facial landmarks in the reference image; and
transforming, based on the landmark difference, the respective subsequent frames.

7. The method of claim 6, wherein the reference image comprises the first image in the sequence of images.

8. The method of claim 6, wherein the reference image comprises an image in which the user's face is oriented such that the user is looking directly at the camera.

9. The method of claim 6, wherein:
determining the landmark difference comprises determining a difference in an orientation of the user's face relative to the camera; and
transforming, based on the landmark difference, the respective subsequent frames comprises transforming a perspective of the subsequent frame to match a perspective of the reference image.

10. The method of claim 6, further comprising determining, in the reference image, a light intensity corresponding to each of the reference landmarks, wherein:
determining the landmark difference comprises determining a difference in light intensity between one or more reference landmarks in the reference image and one or more corresponding facial landmarks in the subsequent frame; and
transforming, based on the landmark difference, the respective subsequent frame comprises transforming the lighting intensity of the subsequent frame to match the lighting intensity of the reference image.

11. The method of claim 1, wherein the extraction and the adjustment are performed by a face alignment model, the face alignment having been selected from a plurality of face-alignment models based on one or more evaluation scores of the face-alignment models.

12. The method of claim 11, wherein the one or more evaluation scores comprise one or more of a circular radius score, a mean offset score, or an impacted pixels score.

13. The method of claim 1, wherein the one or more vital signs of the user comprise one or more of a blood oxygenation, a heart rate, a respiratory rate, or a blood pressure.

14. One or more non-transitory computer readable storage media storing instructions and coupled to one or more processors that are operable to execute the instructions to:
access a video of a user's face captured by a camera, the video comprising a sequence of image frames;
access, for each image frame in the video, (1) one or more facial landmarks of the user's face determined by a facial landmark detection (FLD) model and (2) a corresponding determined position in the image for each facial landmark;
determine, based on the one or more facial landmarks and corresponding positions, a motion of the user's face in the captured video;
extract, from the determined motion of the user's face, a corrected motion signal of the user's face in the video;
adjust, based on the extracted corrected motion signal of the user's face, one or more determined positions of one or more facial landmarks in one or more image frames of the video;
determine, based at least in part on the adjusted positions of the facial landmarks in the sequential images of the video, an rPPG signal; and
determine, based on the determined rPPG signal, one or more vital signs of the user.

15. The media of claim 14, further comprising instructions that are coupled to one or more processors that are operable to execute the instructions to:
define a reference image of the user's face, the reference image comprising one or more reference facial landmarks;
determine, for each of one or more subsequent frames in the sequence of image frames, a landmark difference between the facial landmarks in that frame and the corresponding reference facial landmarks in the reference image; and
transform, based on the landmark difference, the respective subsequent frames.

16. A method comprising:
generating, for each frame in a reference video of a reference subject, a plurality of ground-truth facial landmarks;
accessing, for each frame in a test video of a test subject, a plurality of facial landmark detection (FLD) facial landmarks determined by an FLD model; and
evaluating the FLD model according to one or more scoring criteria applied to each frame of the reference video and each respective frame of the test video.

17. The method of claim 16, wherein the reference subject comprises a mannequin head.

18. The method of claim 17, wherein generating the plurality of ground-truth facial landmarks comprises:
defining an initial position for each ground-truth facial landmark by averaging the positions of each of one or more initial facial landmarks identified in reference video during a predetermined static phase in which the mannequin head is stationary; and
for each subsequent frame in the reference video after the predetermined static phase, applying a predetermined movement, relative to the previous ground-truth position, to each facial landmark to generate its ground-truth facial landmark position for that frame.

19. The method of claim 16, wherein the one or more scoring criteria comprises one or more of a circular radius score, a mean offset score, or an impacted pixels score.

20. The method of claim 16, wherein evaluating the FLD model comprises evaluating a transformed landmark determined by a facial alignment model applied to an output of the FLD model.
